(19) United States
(12) Patent Application Publication
(10) Pub. No.: US 2019/0175725 A1
(43) Pub. Date: Jun. 13, 2019

(54) Prophylactic and Immunomodulatory Compositions and Uses

(71) Applicant: Kherion Technology Limited, Cambridge (GB)

(72) Inventor: Marc Creus, Geneva (CH)

(21) Appl. No.: 16/152,910

(22) Filed: Oct. 5, 2018

Related U.S. Application Data
(63) Continuation of application No. 11/994,050, filed on Jul. 15, 2010, now Pat. No. 10,092,643.

(30) Foreign Application Priority Data
Jun. 30, 2005 (GB) 0513431.7

(51) Int. Cl. A61K 39/39

(52) U.S. Cl. CPC A61K 39/39; A61K 2039/55544

(57) ABSTRACT

Provided is use of a sphingosine compound, or a derivative of a sphingosine compound, in the manufacture of a vaccine effective in the treatment and prevention of an infectious disease and treatment and/or prevention of an autoimmune disease. Also provided is use of a sphingosine compound, or a derivative of a sphingosine compound, in the manufacture of an immunomodulation medicament effective in the prevention of infectious disease and treatment and/or prevention of an autoimmune disease.

The present invention relates to uses of sphingosine derivative compounds, such as D-erythro-sphingosine 1-phosphate (S1P) and related compounds (whether naturally occurring or synthetic) as adjuvants to vaccines and/or immunomodulators in the context of prophylaxis and prevention of disease, including vaccination with live, dead, genetic or subunit vaccines, particularly against tuberculosis, and also for the treatment and prevention of autoimmune diseases, particularly autoimmune diseases triggered or worsened by infection and/or by contact with microorganisms, such as Crohn's disease and sarcoidosis. Such compounds are characterized in that they stimulate macrophagic phospholipase D (PLD), promote phagolysosomal fusion and alter migration and cytokine secretion of immune cells to promote a concerted immune response that improves the outcome of the host upon infectious challenge, leading to a reduction of the infectious burden and amelioration of associated autoimmune complications.

Most particularly, the invention relates to compounds, compositions and methods for the prevention of infection and disease arising from Mycobacteria, especially Mycobacterium tuberculosis (Mtb), for instance by improved antigen presentation of vaccines, such as live mycobacterial vaccines that usually inhibit phagolysosomal fusion.

Immuno-defence mechanisms against infective agents include innate and acquired immunity responses developing during the early phases of the infection and later, respectively. The innate immune system (IIS) is non-specific and mediated by the activity of mononuclear, polymorphonuclear phagocytes and Natural Killer cells, whereas the acquired immune system (AIS) is specific and mediated by T and B lymphocytes, which are clonally distributed and characterised by specificity and memory. Physiologically, the IIS and AIS interact to affect an optimal immune response.

Antigen-presenting cells, such as macrophages and dendritic cells, are fundamental cellular components of IIS. These are able to phagocyte and digest exogenous particles by hydrolytic activity of lysosomal enzymes and exert an immunoregulatory effect on the AIS by means of antigen-presentation and by secretion of cytokine and chemokines, leading to a concerted immune response.

During phagocytosis, firstly exogenous material adheres to the plasmatic membrane of macrophage and then the latter is enveloped within the cytoplasm resulting in a vesicle named a phagosome. This reaches the middle of the cell where it is fused with lysosomes, vacuolar organelles rich in hydrolytic enzymes active at acid pH, 4.5-5.0, thus generating a phagolysosome, a process that is mediated by phospholipase D (PLD) activation. PLD is a membrane enzyme widely diffused within the mammalian cells whose activity is under the control of hormones, neurotransmitters, growth factors and cytokines. During the maturation process of the phagolysosome, mediated through PLD, there is a progressive acidification of the phagosome whose pH decreases from neutral to acid values, namely 5 or less within the phagolysosome, activating metabolic mechanisms whose final goal is to make the macrophage able to eliminate the foreign microorganisms (viruses, bacteria, fungi, protozoa, parasites, etc.).

Some intracellular microorganisms, such as Mycobacterium tuberculosis, the etiological agent of tuberculosis (TB), are able, through various evasion mechanisms, to escape macrophage microbicidal mechanisms, including by inhibition of phagolysosomal fusion. The current most-widely used vaccine against TB, the bacillus Calmette-Guerin (BCG), which consists of a live, attenuated strain of Mycobacterium bovis, also inhibits phagolysosomal fusion in macrophages. The inhibition of phagolysosomal fusion by BCG contributes to its poor antigen-presentation and limits BCG's effectiveness as a vaccine and adjuvant.

Tuberculosis currently represents one of three most diffused diseases in the world together with acquired immunodeficiency syndrome (AIDS) and malaria.

Lack of effective prophylactic measures against TB (e.g. vaccines) is an important factor contributing to the increase of MTB occurrence.

Reports suggest that a successful outcome for the host upon Mtb infection depends largely upon appropriate cellular immunity (i.e. mediated by T cells). There are two broad (possibly overlapping) types of T-cells: Th1 (which secrete IL-2, IFN-gamma and appear to play a protective role in intracellular infections) and Th2 (which secrete Il-4, IL-5 and IL-10). The strength of a Th1 immune response is reported to relate directly to the clinical manifestations of the disease, whereas the strength of a Th2 immune response may exert a negative influence and prevent control of infection. In addition, a humoral immune response (i.e. mediated by B cells or antibodies) may also contribute to the response against TB. An appropriate concerted immune response is necessary to clear the infection effectively as well as crucial to prevent possible negative consequences of the immune response upon infection, such as excessive or chronic inflammation and tissue necrosis, perhaps associated with autoimmunity.

Infections are sometimes found associated to an inappropriate immune response that leads to lasting or chronic inflammation, often with a failure to clear effectively the associated infection, and to autoimmunity. Crohn's disease and sarcoidosis are two examples of autoimmune disease that have been reported as associated with infection, in particular with mycobacterial-like infection, and in which macrophages are recognized as playing an important role. The infection associated with excessive or chronic inflammation may be due to known pathogens, such as mycobacteria, or to other microbes not normally thought to be pathogenic, such as bacteria normally present in the gut.

Sphingolipids, including sphingosine and derivative compounds, like D-erythro-sphingosine 1-phosphate (S1P), are bioactive lipids with immunoregulatory activity. For example, S1P, a naturally-occurring, polar sphingolipid, was shown to increase PLD activity in macrophages, promote phagolysosomal fusion in Mtb-infected macrophages and, given at concentration close to physiologic ones, was reported to induce antimicrobial activity in human macrophages leading to the intracellular killing of TB. When S1P was intravenously injected in MTB-infected mice, S1P significantly reduced mycobacterial growth and pulmonary tissue damage. S1P has thus been suggested as a promising new antimycobacterial drug for treatment of human tuberculosis (Fraziano et al., International Publication Number: WO 03/045365 “IMMUNOREGULATOR COMPOUNDS”).

Sphingolipids, including S1P, also have direct effects on immune cells other than macrophages: when administered to dendritic cells in vitro, S1P has been reported to alter the secretion of cytokine profiles to alter Th1/Th2 responses. In addition, it is recognized that S1P influences cell migration, including migration of dendritic cells and lymphocytes, as well as cell proliferation. S1P has also been reported to inhibit activation and proliferation of T cells (Jin et al., Blood 2003 Vol. 101:4909-4915). In conclusion, sphingolipids, including derivatives of sphingosine such as S1P, are capable of exerting directly a number of various effects on various immune cells.

However, there is still a requirement to provide more effective prophylactics against infectious diseases such as TB, and to provide immunomodulators that aid in preventing and treating such diseases, as well as autoimmune diseases.

It is thus an aim of the present invention to solve the above problems that are presented in the art, and to provide such prophylactics, and immunomodulators. It is also an aim of the present invention to provide vaccines, pharmaceutical compositions and uses which aid in the prevention, and in some cases treatment of certain diseases.

The present invention results from the observation that S1P increases antigen presentation in the context of vaccines (especially in live vaccines by virtue of immunomodulation and by induction of phagolysosomal fusion); that S 1P is able to exert positive and lasting immunoregulatory effects when used as an adjuvant and—preferably in the presence of vaccination (either with a live, dead, genetic or subunit vaccine), but also in some cases in the absence of a vaccine—S1P is able to modulate the immune response to promote a long-lasting concerted immune response that prevents effective infection or re-infection of Mtb, improve the outcome of the host in the event of an infectious challenge, leading to a reduction of the infectious burden and amelioration of associated autoimmune complications.

Whereas it has been suggested that certain sphingosine compounds may be useful in treating particular infectious diseases, either as directly antimicrobial (in the absence of host cells, such as described in U.S. Pat. No. 6,147,118) or through their immunomodulatory activity (e.g. Fraziano et al., International Publication Number: WO 03/045365 “IMMUNOREGULATOR COMPOUNDS”), an original and unexpected aspect of the present invention is that sphingosine compounds can be used in vaccines, for instance in the prevention or prophylactic treatment of infectious diseases. When used as an adjuvant to vaccines, sphingosine compounds may lead to effective or improved immunity and long-lasting immune regulation.

Furthermore, although certain sphingosine compounds have been suggested as potential therapeutics for autoimmune disease (e.g. Yamamura et al, Current Topics in Medicinal Chemistry, 2004, 4:561-567) and have been postulated to be applicable for the treatment of autoimmune disease through their regulatory activity of S1P receptors, particularly in lymphocytes and cells of the acquired immune system, (e.g. Brinkmann et al, 2002, JBC, 277(24):21453-21457 or Goetzl & Rosen, 2004, 114(11):1531-1537; Baumruker et al., International Publication Number: WO 03/097028; Foster, International Publication Number: WO 04/028521 and Buehlmayer et al, International Publication Number: WO 04/024673), a novel and unexpected aspect of the present invention is that sphingosine compounds, in particular sphingosine compounds that increase the antimicrobial activity of cells of the native immune system (e.g. macrophages and dendritic cells), and which may allow long-term immunity in the context of vaccines, can also be used for the treatment of autoimmune diseases, particularly when the autoimmune disease is associated with infection.

Thus, it is a further aim of the present invention is to provide new means for the prevention of infections, whether derived from known pathogens like Mycobacterium tuberculosis or derived from other microbes not normally considered pathogenic, such as gut bacteria, as well as for the prevention and treatment of autoimmune complications associated with these infections, particularly chronic inflammations such as Crohn's disease or sarcoidosis.

Further, the present invention has the object of regulating the immune response to increase the effectiveness of vaccines (including live, dead, genetic and subunit vaccines).

Therefore, the present invention aims to provide compounds to be used as immunomodulators able to induce, restore or increase the efficiency of immuno-defence mechanisms, whether on their own or in combination (e.g. with vaccines), for prophylaxis against infections, principally (but not exclusively) infections resulting from bacteria and specifically Mycobacterium tuberculosis, and also for the prevention and treatment of associated autoimmunity (e.g. Crohn's disease and sarcoidosis).

Such compounds, which are sphingosine derivatives or analogs, like D-erythro-sphingosine 1-phosphate (S1P), are able to stimulate in a dose-dependent effect the macrophage PLD both in presence and absence of infection. In addition, such compounds promote phagolysosomal fusion (thus promoting antigen presentation even of live mycobacterial vaccines, which usually inhibits phagolysosomal fusion) and alter migration and cytokine secretion of immune cells to promote a concerted immune response that improves the outcome of the host upon infectious challenge or in susceptibility to autoimmune disease associated to infection.

An object of the invention is to provide pharmaceutical compositions containing sphingosine derivatives, particularly S1P, and methods for their preparation. These compositions are preferably in the form of solutions, emulsions or suspensions, but can also be microencapsulated or administered (for example, by injection into the bloodstream or by feeding) from cells or organisms (or derivatives thereof) containing S1P (whether live or dead, and whether naturally-occurring or recombinant). These compositions can also be used in combination with other compounds, such as vaccines, either simultaneously with vaccines or at different times.

The invention further provides use of a sphingosine compound, or a derivative of a sphingosine compound, in the manufacture of an immunomodulation medicament effective in the treatment and/or prevention of an autoimmune disease associated with infection.

Thus the present invention makes use of sphingosine compounds and derivatives of such compounds. In the context of the present invention, the term derivatives is intended to include compounds that are structurally related to sphingosine compounds having a similar or the same effect as sphingosine compounds, most particularly sphingosine-1-phosphate, as well as analogues of any such sphingosine compounds or structurally related compounds.

Sphingosine compounds, derivatives and analogues included in the present invention can readily be identified by known assays, such as the assay for therapeutically efficient candidate compounds disclosed in WO 03/045365, the content of which is incorporated herein by reference. This assay can be used to assess the capability of candidate compounds, derivatives or analogues to promote stimulate, increase or accelerate the maturation of phagolysosomes in infected macrophages. The assay comprises the following in vitro procedure:
(a) bringing the macrophages into contact with fluorescein-labelled and phagocytosis-susceptible particles, and successively treating with candidate molecule, compound, derivative, or analogue;
(b) monitoring the fluorescence emission and the maturation of phagolysosomes detected by the decrease of fluorescence emission; and
(c) comparing the decrease in fluorescence emission to the decrease in fluorescence emission detected in the same procedure in the absence of the candidate molecule.

The particles used in the assay may be microorganisms, or synthetic particles and may be labelled with fluorescein isothiocyanate. Preferably the macrophages are brought into contact with bacteria at the multiplicity of infection (MOI) from 0.1:1 and incubated from 1 hour to 7 days at 37° C. After incubation, the macrophages are treated with the substance to be tested, whose activity is evaluated for a period of from 15 to 90 minutes, preferably about 30 minutes. The fluorescence may be monitored, for example, by means of a fluorometer, a flow cytometer, or a fluorescence microscope, detecting the biogenesis of mature phagolysosomes through the emission decrease of fluorescence signal. Decrease in the fluorescence emission is compared with that observed according to the same procedure in the absence of candidate molecule. Any substance able to promote, stimulate, increase or accelerate the maturation of phagolysosomes in macrophages will be detected by being able to modify the emission profile of the fluorimetric signal when compared with the observed model in the absence of the substance.

Specific examples of derivatives and analogues that may be used in the present invention include: include N, N-dimethylsphingosine, DL-threo-dihydrosphingosine, 1-benzyl-3-carboxyazetidine and N-acetylsphingosine (C₂-ceramide), as well as phosphate and phosphonate analogs, such as 3-(N-benzyl or alkyl or alkylamino) aminopropylphosphonic acid. Further such compounds are disclosed in the following published documents, the contents of which are incorporated herein by reference:

J. Med. Chem. 2004 Dec. 30; 47(27):6662-5, “A rational utilization of high-throughput screening affords selective, orally bioavailable 1-benzyl-3-carboxyazetidine sphingosine-1-phosphate-1 receptor agonists.”, Hale J J, Lynch C L, Neway W, Mills S G, Hajdu R, Keohane C A, Rosenbach M J, Milligan J A, Shei G J, Parent S A, Chrebet G, Bergstrom J, Card D, Ferrer M, Hodder P, Strulovici B, Rosen H, Mandala S;

Bioorg. Med. Chem. 2005 May 16; 13(10):3475-85, “Synthesis and evaluation of sphingoid analogs as inhibitors of sphingosine kinases.”, Kim J W, Kim YW, Inagaki Y. Hwang Y A, Mitsutake S, Ryu Y W, Lee W K, Ha H J, Park C S, Igarashi Y;

Bioorg. Med. Chem. Lett. 2005 Feb. 15; 15(4):1115-9, “Synthesis and biological properties of novel sphingosine derivatives.”, Murakami T, Furusawa K, Tamai T, Yoshikai K, Nishikawa M;

J. Biol. Chem. 2005 Mar. 18; 280(11):9833-41, E pub 2004 Dec. 8, “Sphingosine 1-phosphate analogs as receptor antagonists.”, Davis M D, Clemens J J, Macdonald T L, Lynch K R;

Bioorg. Med. Chem. Lett. 2004 Oct. 4; 14(19):4903-6, “Synthesis of benzimidazole based analogues of sphingosine-1-phosphate: discovery of potent, subtype-selective S1P4 receptor agonists.”, Clemens J J, Davis M D, Lynch K R, Macdonald T L; and

Bioorg. Med. Chem. Lett. 2004 May 17; 14(10):2499-503, “Syntheses of sphingosine-1-phosphate analogues and their interaction with EDG/S1P receptors.”, Lim H S, Park J J, Ko K, Lee M H, Chung S K.

Typically the autoimmune disease is a disease that is associated with infection. The infection may be any infection, but is typically an infection leading to chronic inflammation, and may be caused by a known pathogen, such a mycobacterial pathogen, or by a microbe not normally thought to be pathogenic, such as common gut bacteria. The autoimmune disease may be any autoimmune disease, but preferably is selected from Crohn's disease and sarcoidosis.

The invention also provides use of a sphingosine compound, or a derivative of a sphingosine compound, in the manufacture of a vaccine, effective in the treatment and/or prevention of an infectious disease, and further, use of a sphingosine compound, or a derivative of a sphingosine compound, in the manufacture of an immunomodulation medicament, effective in the prevention of an infectious disease.

Although the infectious disease is not especially limited, it is preferred that the infectious disease is caused by one or more viruses, bacteria, fungi, protozoa or parasites. It is especially preferred that the infectious disease is caused by an intracellular pathogen. Typical intracellular pathogens include Mycobacterium tuberculosis, Bacillus anthracis and Listeria monocytogenes.

Further provided by the invention is use of a sphingosine compound, or a derivative of a sphingosine compound, in the manufacture of a vaccine effective in the treatment and/or prevention of cancer. In some embodiments the vaccine may comprise a component derived from an infective agent, for example the vaccine may be a BCG or dendritic vaccine. The cancer may be any cancer, but it is preferred that the cancer is colon cancer.

In all of the above uses of the invention, a preferred embodiment is one in which the sphingosine compound is used as an adjuvant.

The invention also provides a vaccine effective in the treatment and/or prevention of an autoimmune disease, comprising a sphingosine compound, or a derivative of a sphingosine compound. The autoimmune disease is typically one as already described above.

A vaccine is also provided by the invention, which is effective in the treatment and/or prevention of an infectious disease, comprising a sphingosine compound, or a derivative of a sphingosine compound. It is generally preferred that the infectious disease is one as described above. Typically the antigen present is BCG or Mycobacterium tuberculosis, whether live dead or a derivative thereof, and macrophages are treated with D-erythro sphingosine 1-phosphate.

Further provided by the invention is a pharmaceutical composition comprising a prophylactically-effective amount of sphingosine compound, or a derivative of a sphingosine compound, alone or together with one or more pharmaceutically acceptable antigens, adjuvants and/or excipients. The composition may be in any suitable form, depending on patient and disease state, but typically the composition is in the form of solid or solution, emulsion or suspension. In some embodiments it may be in the form of microencapsulated particles, an aerosol, or a liposomal suspension.

Typically, the compound is suitable for administration from cells or organisms or derivatives thereof. These cells or organisms may be live or dead, and may be naturally-occurring or recombinant. The cells or organisms contain the immunoregulatory compounds, and are administered either directly into the host, or indirectly by prior in vitro or ex vivo treatment of cells or tissues.

The composition may be administered in any desired manner, but in preferred embodiments it is administered by injection or oral administration.

The composition may contain the sphingosine compound or derivative in any desirable concentration, depending on patient and disease state, but in some preferred embodiments it contains the sphingosine compound in concentration of from 0.1 μM to 10 mM, more preferably from 0.1 μM to 10 μM .

The composition according to the invention may contain the sphingosine compound as the sole prophylactic or preventative or therapeutically effective agent or in association with other prophylactic or preventative active agents. Preferably, the other prophylactic or preventative active agents include vaccines, whether live, genetic, dead or subunit.

Typically, a prophylactically or preventatively-effective amount of the sphingosine compound is mixed with additional pharmaceutically acceptable substances. The additional pharmaceutically acceptable substances are not limited. In some embodiments they may be selected from surfactants, buffers, stabilisers, preservatives, anti-oxidants, adjuvants, or antigens, including vaccines, whether live, dead or subunit.

The sphingosine compound is not especially limited, provided that its prophylactic and/or therapeutic function is not impaired. Typically it is selected from D-erythro or L-threo sphingosine isomers, mono-, di- or tri-phosphates of sphingosine, and natural or synthetic analogs and derivatives of sphingosine capable of mimic their immunoregulating activity. The immunoregulating activity is not especially limited, and, for example, may be associated with the induction of phagolysosome maturation in macrophages or epithelial cells upon encountering an antigen. Alternatively, it may be associated with the activity increase of macrophage phospholipase D upon encountering an antigen, with the activity increase in cytokine secretion, the production increase of oxygen and/or nitrogen reactive intermediates in antigen-presenting cells, or the expansion of specific T cells against the antigens presented by antigen-presenting cells. In this case the antigen may be selected from a microbe or vaccine. The antigen presenting cells may be selected from dendritic cells, macrophages and monocytes.

Generally, the sphingosine compound is a compound having the following structure, or a salt or other derivative of this compound:

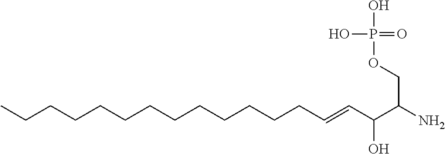 [SMILES: CCCCCCCCCCCCC/C=C/C(O)C(N)COP(=O)(O)O]

The most preferred sphingosine compound is D-erythro-sphingosine 1-phosphate (S1P), or a salt or other derivative of S1P.

The invention also provides a prophylactic method against an autoimmune disease or an infectious disease, which method comprises vaccinating a subject with a vaccine as defined above, or administering a pharmaceutical composition as defined above.

Further provided is a method to induce or restore or increase immunoregulatory activity which method comprises vaccinating a subject with a vaccine as defined above, or administering a pharmaceutical composition as defined above.

These methods may comprise administration of the vaccine or the pharmaceutical composition simultaneously, sequentially or separately to one or more antigens and/or excipients.

If further antigens and/or excipients are present, they may be selected from vaccines, whether live, dead, genetic or subunit, and allergens.

Typically the antigen present is BCG or Mycobacterium tuberculosis, whether live dead or a derivative thereof, and macrophages are treated with D-erythro sphingosine 1-phosphate.

S1P causes phagolysosomal fusion in BCG/mycobacterial infection (Garg et al., 2004, JID Vol. 189, pp. 2129 to 2137), leading to better antigen presentation, particularly useful in live vaccines. Because phagolysosomal fusion helps to eradicate infections, and because infection often contributes to inflammation in autoimmune disorders, S1P may be clinically useful in the treatment of autoimmune diseases, such as Crohn's disease (reported to be associated with Mycobacterium avium paratuberculosis infection) or sarcoidosis (reported to be associated with tuberculosis or a viral infection).

S1P is an immunoregulator and macrophage activator, which alters cytokine secretion of immune cells. This characteristic is particularly useful in dead or subunit vaccines, because an altered cytokine profile upon vaccination, for example minimizing local IL4 cytokine secretion (associated with a Th2 response), can increase the effectiveness of a TB-booster vaccine. Altering the local Th1/Th2 response may also reduce inflammation in the case of autoimmune disorders.

S1P reduces T-cell motility and prevents inflammatory responses at the site of infection. Stopping lymphocyte infiltration at a site of inflammation helps reduce necrosis in the lungs of a TB model (Garg et al., 2004, JID Vol. 189, pp. 2129 to 2137); further, S1P administered to mycobacterial-infected macrophages promotes phagolysosomal fusion (BCG vaccine also prevents fusion, which can be reversed with S1P); by association, the same characteristics may reduce inflammation and necrosis in autoimmune disorders, such as commonly found in the gut of Crohn's patients, particularly when bacteria infiltrating the gut tissues causes prolonged inflammation. Temporary prevention of T-cell infiltration also alters the outcome of vaccination: for example, in the case of TB vaccination this may lead to regulated antigen presentation and to increased immunity.

Immunomodulating compounds according to the present invention are sphingosine derivatives and analogs (whether naturally occurring or synthetic). Sphingosine is a long chain amino alcohol constituent of cellular membranes and together with sphingosine-1-phosphate is a member of a class of second lipid messengers produced in response to growth factors. Sphingosine itself and its derivatives can be both in erythro and threo conformation, (D) and (L) configuration and cis and trans across the double bond. Sphingosine derivatives and their analogs are also able to mimic immunomodulating activity, particularly those selected among D-erythro or L-threo and monophosphates, diphosphates, triphosphates thereof and more particularly D-erythro sphingosine 1-phosphate (S1P).

Administration of sphingosine derivatives, even before infectious challenge, induces an increase of microbicidal activity, particularly bactericidal and specifically mycobactericidal, upon subsequent infection of both animal and human macrophages and epithelial cells, which can be detected as a decrease of colony forming pathogen units. Such an immunomodulating activity is associated to the induction of phagolysosome maturation and increase of the activity of macrophage phospholipase D in pathogen infected cells and increase of the production of oxygen and/or nitrogen reactive intermediates. Although the infectiveness of Mtb in epithelial cells is not as high as in macrophages, epithelial cells are important in preventing Mtb from further infiltration and therefore, form a “line of defence” against spread of infection. S1P administered before infection increases the capacity of macrophages and epithelial cells to control an infection and therefore S1P may prove a useful prophylactic against further infection and prevent dissemination of pathogenic bacilli or of the normal gut flora in animals and humans, particularly in the gut.

Administration of S1P to TB-infected macrophages induces expansion of Mtb specific T cells, demonstrating the capacity of S1P to promote long-lasting anti-mycobacterial immunoregulation, which involves cell-mediated immunity, a finding that can be applied in the context of disease prevention and prophylaxis and, specifically for improving vaccine efficiency.

Administration of S1P in mice at levels in which immunoregulatory activity can be measured appears safe, suggesting that S1P may be administered safely directly in animals or humans to exert immunoregulatory prophylactic, therapeutic or vaccine-enhancing activity.

Therefore the above sphingosine derivative compounds to be used as immunomodulators and for related uses in the preparation of medicaments and vaccines with immunoregulating activity, e.g. associated to the above phenomena, are object of the present invention.

Sphingosine derivatives as immunoregulating compounds according to the present invention can be used advantageously in the prophylaxis and prevention of infections resulting from any pathogen as viruses, bacteria, fungi, protozoa and other parasites and particularly Mycobacterium tuberculosis, Bacillus anthracis or Listeria monocytogenes.

These compounds can also be used advantageously to prevent and treat autoimmune disease associated to infections and particularly Crohn's disease and sarcoidosis.

The present invention also provides pharmaceutical compositions comprising prophylactically or therapeutically active amounts of a sphingosine derivative compound together with one or more pharmaceutically acceptable antigen, adjuvant and/or excipient (including vaccines).

These compositions can contain the immunoregulating compound as the sole active agent when used in a treatment for simple promotion of native or acquired immunodefences or can contain the same in association with other prophylactic (such as vaccines), anticancerous or microbicidal medicaments or compositions.

Pharmaceutical compositions according to the present invention can be formulated according to known methods that include the presence of a pharmaceutically acceptable vehicle both in solid and liquid form. Examples of these vehicles and formulation methods are reported in specialised literature. Pharmaceutical compositions suitable for immunoregulating activity must contain an effective amount of the active principle according to the present invention. In the case of liquid compositions such an amount corresponds to 0.1 microM to 10 mM, preferably from 1 to 200 microM, which are concentrations present in the medium in cells grown in vitro at which effects can be observed, and the concentration of which 100 microlitres is effective per mouse when administered intravenously. In the case of solid compositions the amount expressed as micrograms corresponding to the above concentrations can be used. Pharmaceutical compositions according to the present invention are administered to a subject (or to cells in vitro or ex-vivo) in such amount that is effective for successful prevention or treatment of the related disease. Relative amount can be varied depending on a multiplicity of factors like individual conditions, weight, sex and age, as well as method and site of administration (e.g. larger amounts may be administered in a targeted manner, for example directly into the gut by oral administration to prevent infection, since the concentration of administered compound may not rise systemically). Preferred formulations are liquid compositions in the form of solutions, suspensions or emulsions to be administered by oral, inhaled (e.g. via aerosol) or parenteral route; other formulations include microencapsulation and also administration (for example, by injection or feeding) from cells or organisms (or derivatives thereof, whether live or dead, and whether naturally-occurring or recombinant) containing the immunoregulatory compounds, either directly into the host, or indirectly by prior in vitro or ex vivo treatment of cells or tissues. These compositions can also be used in combination with other compounds, such as vaccines, either simultaneously with vaccines or at different times. Due to the chemical characteristics of sphingosine derivatives the inclusion thereof within liposomes or emulsions is particularly preferred. According to the present invention the immunoregulating compound can be administered typically in mixture with suitable pharmaceutical diluents, excipients or vehicles conveniently selected by taking in consideration the desired administration route.

Because the inventive compounds are soluble in hydro-alcoholic solvents (water/ethanol), such solutions thereof are used as such, if suitable, or are used as starting reagent for the preparation of different pharmaceutical formulations. Further, the composition can contain pharmaceutically acceptable optional additives as surfactants, buffers, stabilisers, preservatives and antioxidants.

The preparation of the pharmaceutical compositions according to the invention includes methods known in the specific field. The immunomodulating compounds are mixed in solid or liquid form with suitable excipients, possibly with any desired optional additive, preferably in hydroalcoholic solutions, at concentrations from 0.1 microM to 1.0 mM, preferably from 0.5 microM to 1 mM or, in the case of solid compositions, at corresponding weight amounts. These solutions are then formulated in liquid compositions suitable to be administered, as solutions, suspensions, emulsions, or liposome-encapsulated (or other encapsulation method, such as in calcium alginate) or dried as powders or granules suitable to be formulated in solid compositions. In the case of liquid compositions 0.1 microM to 10 mM, preferably from 1 to 200 microM, are concentrations present in the medium in cells grown in vitro at which effects can be observed and the concentration of which 100 microlitres is effective per mouse when administered intravenously.

The present invention also provides an in vitro method to induce or restore or increase the immunoregulatory activity of immune cells, preferably phagocytes, such as human macrophages, dendritic cells and mast cells. Immune cells cultured as described in the experimental section and treated with relevant antigens, such as vaccines (whether live, dead, genetic or subunit), susceptible to phagocytosis, for example at a pathogen/macrophage infection ratio (MOI) from 1:1 to 10:1 for a period from 1 to 24 hours are treated with solutions containing sphingosine derivatives according to the invention at concentrations from 0.1 μM to 10 mM, preferably from 1-200 μM, particularly from 1-10 μM, such as about 5 μM over an incubation period from 1 minute to 24 hours, preferably from 15 to 90 minutes. Suitable antigens are viruses, bacteria, fungi, protozoa, parasites, particularly Mycobacterium tuberculosis and, in particular, vaccines such as BCG, cell extracts and subunit vaccines. Immune cells, such as macrophages, are preferably treated with D-erythro sphingosine 1-phosphate.

The invention will be further illustrated by way of example only, with reference to the following specific embodiments, which describe reagents, conditions and procedure suitable for the practice of the invention.

### EXAMPLE 1 S1P+BCG TO PREVENT TB

BCG is the most widely used vaccine against tuberculosis, but it is ineffective in producing long-lasting effective immunity and it is particularly ineffective in preventing TB in adults (Doherty, Tropical Medicine and International Health, Vol. 9 No 7, pp. 818-826). The cause of this lack of effectiveness may be related to a failure of correct antigen presentation, because BCG, like virulent Mtb, prevents lysosomal fusion occurring in macrophages, which are important antigen-presenting cells. In addition, it is known in the art that immunoregulators may improve vaccination-mediated immunity, such as reported upon BCG vaccination in combination with oligodeoxynucleotides containing cytidine phosphate guanosine (CpG) motifs (Freidag et al, 2000, Infect. Immun., Vol 68, pp. 2948-53). It is relevant that activation of sphingosine kinase, the primary enzyme for S1P production in cells, is able to induce IFN-gamma production in Th1 cells (Yoshimoto et al, 2003, Journal of Immunology, pp. 1352-1359), because IFN-gamma production and a Th1-mediated response is known to be associated with effective antimycobacterial immunity (Raja, 2004, Indian J Med. Res., Vol. 120, pp. 213-232).

The present invention relates to sphingosine compounds such as S1P given as an adjuvant to vaccines, in particular to a live BCG vaccine, in which S1P increases antigenicity of the vaccine and, by its immunoregulatory activity, induces improved long-lasting vaccine-immunity.

In particular, increased prophylaxis against TB infection using a live vaccine in combination of S1P may be achieved in the following way: in mice, for example BALB/c mice, a total of 0.1 to 200 nanomoles of S1P, preferably 1 to 20 nanomoles per mouse, can be added and mixed with prophylactic amounts of BCG vaccine (amounts of BCG required in well known in the art), prior to inoculation with the mixture.

In general, the efficacy of sphingosine compounds of the present invention (such as sphingosine-1-phosphate) as an adjuvant for a Bacillus Calmette-Guerin (BCG) vaccine can be tested using the following protocols. In particular, the protocol aims to test that:
a) BCG immunogenicity is increased when the sphingosine compound (e.g. S1P) is given as an adjuvant in mice, as measured by immunological markers such as cytokine responses, cell proliferation and antibody production, including dose responses to S1P
b) Protection against virulent tuberculosis is improved when using the sphingosine compound (e.g. S1P) as an adjuvant to BCG (compared to BCG alone), as measured upon challenge with virulent TB in mice, including measurements of mice survival rates, extent of bacillus survival in spleen and lungs and histology for amount of inflammation in tissues.
In the following, S1P is used as an example of a sphingosine compound to be tested.

Phase 1—Testing BCG Immunogenicity
The BCG vaccine and S1P are given to 5 or 6 week-old C57BL/6 mice, normally intravenously (or alternatively as subcutaneous or intradermal injection). Group mice according to the following variables:
   Saline control (no vaccination)
   Mixture of a normal dose of BCG+0, 1, 5, 20 nmoles of S1P (as adjuvant)—the hydrophobicity properties of S1P ensure binding to the surface of the live vaccine, even in the presence of small amounts of detergent usually added to the vaccine to achieve a suspension of cells.

At weeks 1, 3 and 8, carry out the following analyses:
Peripheral blood, spleen and lung lymphocyte cultures, for cytokine measurements by ELISA (for method details: Holten-Andersen et al., Infection and Immunity, 2004, Vol 72, No. 3, p. 1608-1617). Measure cytokines expressed upon PPD stimulation.
   IFN-gamma
   TNF-alpha
   IL2, IL4, IL10, IL12
   FACS analyses of lymphocytes from vaccinated and control mice, isolated from lungs. Culture cells in the presence of PPD for 72 hours before staining (+control wells without antigen). Measure CD4, CD8 cells and intracellular IFN-gamma (see: Holten-Andersen et al., Infection and Immunity, 2004, Vol 72, No. 3, p. 1608-1617).
   Histological analysis of lungs
   At week 8 (prior to TB infection), also measure BCG CFU in spleen and lungs

Phase 2—Testing, BCG/S1P Protection

At week 8 post-vaccination, infect the remaining mice with M.tuberculosis (Erdman) cultured from infected organs (Andersen et al. Infect. Immun., 1991, Vol. 59, p 1558-1563). Use aerosol infection with inoculum of 30-40 CFU per mouse (see: Holten-Andersen et al., Infection and Immunity, 2004, Vol 72, No. 3, p. 1608-1617), or a higher dose of intravenous, subcutaneous or intradermal.

Survival rates of infected mice are measured, monitoring weight loss and difficulty of breathing.

A subset are sacrificed at week 6 after TB infection, for detailed analysis:
CPU counts: Mycobacterial cultures from lungs grown on 7H11 plates, containing 2-triophenecarboxylic acid anhydride (to selectively inhibit BCG growth)
Histological analysis of lungs

When following the above protocols using an appropriate number of mice and experiments for appropriate statistical analysis, it can be demonstrated that S1P itself has novel, unexpected and advantageous properties as a vaccine adjuvant, as determined by some of the parameters measured.

Experimental methods similar to some of those described above for testing of the efficacy of an adjuvant may additionally be described in more detail the following references:
Garg et al., JID, 2004, Vol 189, p 2129-38
Holten-Andersen et al., Infection and Immunity, 2004, Vol 72, No. 3, p. 1608-1617
Hsieh et al, Vaccine, 2004, Vol 22, p 655-659
Andersen et al., Infect. Immun., 1991, Vol. 59, p 1558-1563
Hovav et al, Infection and Immunity, 2005, Vol 73, No. 1, p 250-257

BCG has also been used in the context of cancer treatment (Uyl-de Groot et al, 2005, Vaccine, 23, pp. 2379-2387). In the present invention, S1P is revealed to be effective as an immunomodulator of vaccines, such as BCG and dendritic vaccines, for the treatment of cancer, for example colon cancer.

EXAMPLE 2 S1P+TO DEAD OR SUBUNIT VACCINES TO PREVENT TB

It is known that the immunogenicity and protective efficacy of subunit vaccines may be increased by lipid adjuvants, for example monophosphoryl lipid A-trehalose dicorynomycolate (Ribi), which improves vaccine immunity conferred by TB protein antigens (Hovav et al 2005, Infection and Immunity Vol. 73, No 1, pp. 250-557). Moreover, it has been reported that successful TB vaccines may need to be immunoregulatory rather than merely Th1 boosting (Rook et al, 2005, Vaccine, Vol. 23, pp. 2115-2120).

The present invention relates to S1P as a novel immunoregulatory adjuvant to dead or subunit vaccines. In particular, increased prophylaxis against TB infection using a subunit or dead vaccine in combination of S1P may be achieved in the following way: in mice, for example BALB/c mice, a total of 0.1 to 200 nanomoles of S1P, preferably 1 to 20 nanomoles per mouse, can be added and mixed with prophylactic amounts of dead or subunit vaccine prior to inoculation with the mixture. In the case of an anti-mycobacterial vaccine, antigens may be cell-culture filtrates of mycobacteria or recombinant antigens, such as ESAT-6, 85B or CFP21, all administered in prophylactically-effective amounts, which are known in the art (for example, 5 micrograms of each antigen per mouse). Alternative methods of vaccine and or S1P administration can also be used, as described in example 1 (above).

For instance, in alternative embodiments S1P can be given orally, for example as an additive in the diet or by gastric infusion, to increase the levels of S1P found in plasma, preferably to above 200 nM, most preferably above 600 nM. As a further alternative, S1P can be given by aerosol administration directly into the lungs, preferably to final concentrations equivalent to above 1 μM, as suggested by measurements of S1P in bronchoalveolar lavages of non-TB patients: aerosol administration increases mucosal immunity and methods are known in the art, for example by microencapsulation with calcium alginate beads or as described by Carpenter et al, (2005, Journal of Controlled Release, Vol. 104, pp. 67-77). In addition, S1P can be administered as a “classical” immunoregulatory adjuvant—i.e. together and simultaneously with the vaccine—or separately, but at a time proximal to the time of BCG vaccination, preferably within a period of 1 week before or after vaccination.

EXAMPLE 3 S1P TO TREAT OR PREVENT CROHN'S DISEASE

Crohn's disease is an autoimmune disease characterized by a chronic inflammatory process in the gut, in which CD4+ T-cells represent the vast majority of activated mononuclear cells infiltrating the gut. There is evidence in the literature that CD4+ T-cells play a key role in the pathogenesis of tissue damage in Crohn's disease (Monteleone et al, 2002, Gut, Vol. 50, Suppl. III, pp. 11160-64). There is also evidence that infections, particularly Mycobacterium paratuberculosis infections, either induce autoimmune disease or contribute to the pathology Crohn's disease (Sartor et al, 2003, Curr. Opin. Gastroenterol., Vol. 19, No 4, pp. 358-365) and, therefore, eradication of infection may alleviate the disease. There is also clear evidence that the intestinal microbiota serves as a trigger for intestinal inflammation and that rapid and efficient killing by macrophages prevents further immune involvements, which may lead to disease (Kaiserlian et al., 2005, J Leukoc. Biol., May 13; Epub ahead of print). Moreover, there is ample evidence that cytokines participate in disease development in other mouse models of colitis, such as in SAMP1/YitFc mice (Bamias et. al, 2005, Gastroenterology, Vol. 128, No 3, pp 654-666). In addition, a mouse model of colitis (trinitrobenzene sulfonic acid—TNBS-induced colitis) can be treated with the immunomodulator compound glatiramer acetate (Aharoni et al, 2005, Inflamm. Bowel Disease, Vol. 11, No. 2, pp. 106-115), suggesting that other immunomodulators may also be useful for treatment.

S1P, when injected intravenously in mice, is known in the art to induce antimycobacterial activity in experimental tuberculosis infection, and therefore is already known as a potential therapeutic agent against infectious disease, particularly against TB. It is an object of the present invention that S1P is not just able to treat infectious disease, but also able to prevent microbial disease infecting an individual, and may be administered to induce effective prophylaxis. A gene involved in the production of S1P (sphingosine kinase 2) is, considering the effectiveness of S1P in preventing TB, a strong candidate (one of approximately 60 genes) for conferring genetic susceptibility to TB in mice, because it is located within a genetic susceptibility locus (Tlr-3). This finding suggests that levels of S1P prior to infection determine the outcome of TB infection and that abnormalities in sphingosine metabolism, particularly in S1P metabolism, are associated with increased susceptibility to infection and a weak long-term immunity against microbes such as Mtb, which may lead to chronic inflammation, as seen in tuberculosis and Crohn's disease.

The present invention relates to the use of S1P as an immunomodulator in the prevention and treatment of autoimmune diseases, such as inflammatory bowel diseases and, in specific, of Crohn's disease. In particular, prevention and/or treatment of inflammatory bowel disease (IBD) with S1P can be achieved in the following way: in an appropriate animal model of IBD, such as in TNBS-induced colitis mouse model or in SAMP1/YitFc mice, increasing gut levels of S1P will prevent and/or ameliorate the signs and symptoms of gut pathology. S1P in the gut may be increased by injecting intravenously, for example once a week, a total of 0.1 to 200 nanomoles of S1P, preferably 1 to 20 nanomoles per mouse. Alternatively, S1P can be administered directly into the gut, for example via oral administration or as an additive in the diet, or by gastric infusion, to increase the local levels of S1P in gut tissues. S1P induces macrophages to clear infections in the gut that participate in pathology and reduces inflammation by preventing T cells from infiltrating the gut.

### EXAMPLE 4 S1P TO TREAT OR PREVENT SARCOIDOSIS

Sarcoidosis is an autoimmune disease most commonly affecting the lung, characterized by a marked Th1 activation (Semenzato et al, 2002, Current Opinion in Pulmonary Medicine, Vol. 8, pp. 441-444). Recent findings suggest that the etiology of systemic sarcoidosis is linked to genetically determined enhanced Th1 immune responses to a limited number of microbial pathogens, including specifically M. tuberculosis (Moller & Chen, 2002, Current Opinion in Pulmonary Medicine, Vol. 8, pp. 429-434).

In one aspect the present invention relates to the use of sphingosine compounds such as S1P as immunomodulators in the prevention and treatment of autoimmune diseases and, in particular, of sarcoidosis. Because sarcoidosis presents a pathology akin to TB and autoimmune disease, prevention and treatment of sarcoidosis can be carried out according to similar methods as described in examples 1, 2 and 3 (above), using an appropriate animal model, such as the murine berylliosis model (Pfeifer et al, 1994, Int. Arch Allergy Immunol. Vol. 104, No 4:332-9) or murine listeriosis model (Mielke et al, 1997, Immunol Rev. Vol. 158, pp. 79-93).

1-48. (canceled)

49. A vaccine for the treatment of a cancer, comprising
a vaccine composition comprising cells derived from a cancer and bacillus Callmette-Guerin (BCG) cells; and
a sphingosine composition comprising a sphingosine compound or a derivative of a sphingosine compound, in an amount effective as an immunomodulator of the vaccine composition.

50. The vaccine of claim 49, wherein the cells are derived from a colon cancer.

51. The vaccine of claim 49, wherein the sphingosine composition comprises any one or more of a D-erythro sphingosine isomer, L-threo sphingosine isomer, mono-phosphate of sphingosine, di-phosphate of sphingosine, and a tri-phosphate of sphingosine.

52. The vaccine of claim 49, wherein the sphingosine composition comprises any one or more of N,N-dimethylsphingosine, DL-threo-dihydrosphingosine, 1-benzyl-3-carboxyazetidine, N-acetylsphingosine (C2-ceramide), and 3-(N-benzyl or alkyl or alkylamino) aminopropylphosphonic acid.

53. The vaccine of claim 49, wherein the sphingosine compound has the following structure: 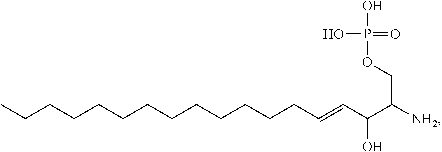 [SMILES: CCCCCCCCCCCCC/C=C/C(O)C(N)COP(=O)(O)O]
or a salt or derivative thereof.

54. The vaccine of claim 49, wherein the sphingosine compound is D-erythro-sphingosine 1-phosphate (S1P), or a salt or derivative thereof.

55. The vaccine of claim 49, further comprising one or more of a pharmaceutically acceptable antigen, pharmaceutically acceptable adjuvant, and a pharmaceutically acceptable excipient.

56. A method comprising
identifying a subject having a cancer;
administering to the subject a vaccine composition comprising cells derived from the cancer and bacillus Callmette-Guerin (BCG) cells; and
administering to the subject a sphingosine composition comprising a sphingosine compound or a derivative of a sphingosine compound, in an amount effective as an immunomodulator of the vaccine composition.

57. The method of claim 56, wherein the cancer is colon cancer.

58. The method of claim 56, wherein the sphingosine composition comprises any one or more of a D-erythro sphingosine isomer, L-threo sphingosine isomer, mono-phosphate of sphingosine, di-phosphate of sphingosine, and a tri-phosphate of sphingosine.

59. The method of claim 56, wherein the sphingosine composition comprises any one or more of N,N-dimethyl sphingosine, DL-threo-dihydrosphingosine, 1-benzyl-3-carboxyazetidine, N-acetylsphingosine (C2-ceramide), and 3-(N-benzyl or alkyl or alkylamino) aminopropylphosphonic acid.

60. The method of claim 56, wherein the sphingosine compound has the following structure: 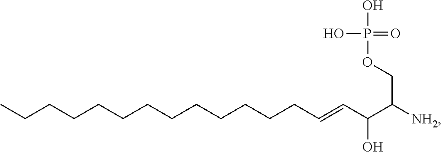 [SMILES: CCCCCCCCCCCCC/C=C/C(O)C(N)COP(=O)(O)O]
or a salt or derivative thereof.

61. The method of claim 56, wherein the sphingosine compound is D-erythro-sphingosine 1-phosphate (S1P), or a salt or derivative thereof.

62. The method of claim 56, further comprising administering one or more of a pharmaceutically acceptable antigen, pharmaceutically acceptable adjuvant, and a pharmaceutically acceptable excipient.
